(19) United States
(12) Patent Application Publication
(10) Pub. No.: US 2009/0130034 A1
(43) Pub. Date: May 21, 2009

(54) RE-ENGINEERED UV DAMAGE ENDONUCLEASE, COMPOSITIONS AND METHODS

(75) Inventors: Paul Doetsch, Atlanta, GA (US); Binwei Song, Lawrenceville, GA (US)

Correspondence Address: GREENLEE WINNER AND SULLIVAN P C, 4875 PEARL EAST CIRCLE, SUITE 200, BOULDER, CO, 80301, US

(73) Assignee: Emory University, Atlanta, GA (US)

(21) Appl. No.: 12/088,310

(22) PCT Filed: Oct. 7, 2005
(86) PCT No.: PCT/US05/36021

§ 371 Date: Jul. 16, 2008

Related U.S. Application Data
(60) Provisional application No. 60/721,022, filed on Sep. 27, 2005.

(51) Int. Cl. A61K 38/43; A61Q 17/04; C12N 15/11; C12N 5/06; C12P 21/04

(52) U.S. Cl. 424/59; 424/94.1; 536/23.2; 435/325; 435/69.1

(57) ABSTRACT

Provided are methods and compositions for reducing damage to skin by ultraviolet light and other agents that cause distortion to double stranded DNA and methods for reducing damage to other organs due to such DNA distortion. These compositions comprise a novel truncated UV damage endonuclease (a truncated derivative of Uvde 1 (UVDE, Uvelp) of Schizosaccharomyces pombe) in conjunction with a cell penetrating peptide, together with components suitable for topical application or other administration to a human or animal in need of treatment to reduce damage to due distortion of double-stranded DNA. Methods for reducing DNA distortion-induced damage or deterioration of condition comprise the step of administering a composition comprising the truncated Uvde 1 of the present invention in conjunction with a fused cell penetration peptide or with a noncovalently bound cell penetration peptide to the skin or other organ, or by other route of administration. Compositions for topical application are also useful for cosmetic or cosmeceutical use.

### CROSS REFERENCE TO RELATED APPLICATIONS

This application claims benefit of U.S. Provisional Application 60/721,022, filed Sep. 27, 2005.

### REFERENCE TO SEQUENCE LISTING, A TABLE, OR A COMPUTER PROGRAM LISTING COMPACT DISK APPENDIX

SEQ ID NO:1, Tat-derived cell penetration peptide sequence

SEQ ID NO:2, Tat-derived cell penetration peptide sequence

SEQ ID NO:3, Cell penetration peptide sequence derived from Drosophila

Antennapedia Protein

SEQ ID NO:4, Cell penetration peptide sequence derived from human Clock protein

SEQ ID NO:5, Cell penetration peptide sequence derived from hPer1 protein

SEQ ID NO:6, Cell penetration peptide sequence derived from hPer2 protein

SEQ ID NO:7, Cell penetration peptide sequence derived from Tat protein

SEQ ID NO:8, Cell penetration peptide sequence

SEQ ID NO:9, Nucleotide sequence encoding truncated Uvde1 protein with hexahistidine tag at C-terminus

SEQ ID NO:10, Truncated Uvde1 protein with hexahistidine tag at C-terminus

SEQ ID NO:11, Nucleotide sequence encoding truncated Uvde1 protein with hexahistidine tag at C-terminus and cell penetration peptide sequence at N-terminus

SEQ ID NO:12, Truncated Uvde1 protein with hexahistidine tag at C-terminus and cell penetration peptide sequence at N-terminus

### BACKGROUND OF THE INVENTION

The field of the present invention is the area of DNA repair enzymes. In particular, the invention concerns the identification of stable ultraviolet DNA endonuclease polypeptide fragments, their nucleotide sequences and recombinant host cells and methods for producing them and for using them in DNA repair processes.

The integrity of its genetic material must be maintained in order for a biological species to survive. However, DNA is continuously subject to damage by endogenous and exogenous agents that can lead to mutations, neoplasia or cell death (Smith et al. 1996. Biochemistry 35:4146-4154; Brash et al. 1991. Proc. Natl. Acad. Sci. USA 88:10124-10128). One potential source of mutations is nucleotide misincorporation by DNA polymerases during DNA replication or repair. In addition, primer/template slippage can occur at repetitive DNA sequences during replication, resulting in single-stranded loops of one or more unpaired bases called insertion/deletion loops (IDLs) that can be mutagenic (Sancar, A. 1999. Nat. Genet. 21:247-249). The human genome has an abundance of simple repeat sequences that are relatively unstable (Petruska et al. 1998. J. Biol. Chem. 273(9):5204-5210). Expansions of such repeat sequences have been associated with human genetic diseases including Huntington's disease, fragile X syndrome and myotonic dystrophy (Pearson et al. 1998. Nucleic Acids Res. 26(3):816-823).

The Escherichia coli Mut HLS pathway has been extensively characterized and is the prototypical DNA mismatch repair (MMR) pathway. This repair pathway recognizes and repairs small IDLs and all single base mismatches except C/C in a strand-specific manner (Modrich, P. 1991. Annu. Rev. Genet. 25:229-253). Mismatch repair pathways have been highly conserved during evolution (Modrich and Lahaue 1996. Annu. Rev. Biochem, 65:101-133). Eukaryotes including Saccharomyces cerevisiae and humans have several genes encoding proteins homologous to bacterial MutL and MutS (Sancar, A. 1999. supra). For example, there are six MutS (Msh1-Msh6) and four MutL (MLH1-3, PMS1) homologs in S. cerevisiae (Kolodoner, R. 1996. Genes Dev. 10:1433-1442). The Msh2p-Msh6p heterodimer binds base mismatches and small IDLs whereas the Msh2p-Msh3p heterodimer binds only small and large IDLs (Marsischky et al. 1996. Genes Dev. 10(4):407-420). A considerable amount of evidence implicates mismatch repair in stabilizing repetitive DNA sequences (Marsischky et al. 1996. supra; Fujii et al. 1999. J. Mol. Biol. 289:835-850; Strand et al. 1993. Nature 365:274-276).

Cellular exposure to ultraviolet radiation (UV) results in numerous detrimental effects including cell death, mutation and neoplastic transformation. Studies indicate that some of these deleterious effects are due to the formation of two major classes of bipyrimidine DNA photoproducts, cyclobutane pyrimidine dimers (CPDs) and (6-4) photoproducts (6-4 PPs). (Friedberg et al. 1995. In: DNA Repair and Mutagenesis, Am. Soc. Microbiol., Washington, D.C., pp. 24-31).

Organisms have evolved several different pathways for removing CPDs and 6-4 PPs from cellular DNA (Friedberg et al. 1995. supra; Brash et al. 1991. supra). These pathways include direct reversal and various excision repair pathways which can be highly specific or nonspecific for CPDs and 6-4 PPs. For example, DNA photolyases specific for either CPDs or 6-4 PPs have been found in a variety of species and restore the photoproduct bases back to their original undamaged states (Rubert, C. S. 1975. Basic Life Sci. 5A:73-87; Kim et al. 1994. J. Biol. Chem. 269:8535-8540; Sancar, G. B. 1990. Mutat. Res. 236:147-160). Excision repair has been traditionally divided into either base excision repair (BER) or nucleotide excision repair (NER) pathways, which are mediated by separate sets of proteins but which both are comprised of DNA incision, lesion removal, gap-filling and ligation reactions (Sancar, A. 1994. Science 266:1954-19560; Sancar and Tang. 1993. Photochem. Photobiol. 57:905-921). BER N-glycosylase/AP lyases specific for CPDs cleave the N-glycosidic bond of the CPD 5′ pyrimidine and then cleave the phosphodiester backbone at the abasic site via a β-lyase mechanism, and have been found in several species including T4 phage-infected Escherichia coli, Micrococcus luteus, and Saccharomyces cerevisiae (Nakabeppu et al. 1982. J. Biol. Chem. 257:2556-2562; Grafstrom et al. 1982. J. Biol. Chem. 257:13465-13474; Hamilton et al. 1992. Nature 356:725-728). NER is a widely distributed, lesion non-specific repair pathway which orchestrates DNA damage removal via a dual incision reaction upstream and downstream from the damage site, releasing an oligonucleotide containing the damage and subsequent gap filling and ligation reactions (Sancar and Tang. 1993. supra).

An alternative excision repair pathway initiated by a direct acting nuclease which recognizes and cleaves DNA containing CPDs or 6-4 PPs immediately 5′ to the photoproduct site has been described (Bowman et al. 1994. Nucleic. Acids Res. 22:3026-3032; Freyer et al. 1995. Mol. Cell. Biol. 15:4572-4577; Doetsch, P. W. 1995. Trends Biochem. Sci. 20:384-386; Davey et al. (1997) Nucleic Acids Res. 25:1002-1008; Yajima et al. 1995. EMBO J. 14:2393-2399; Yonemasu et al. 1997. Nucleic Acids Res. 25:1553-1558; Takao et al. 1996. Nucleic Acids Res. 24:1267-1271). The initiating enzyme has been termed UV damage endonuclease (UVDE, Uve1p, now also termed Uvde1). Homologs of Uvde1 have been found in Schizosaccharomyces pombe, Neurospora crassa and Bacillus subtilis (Yajima et al. 1995. supra; Yonemasu et al. 1997. supra; Takao et al. 1996. supra). The Uvde1 homologs from these three species have been cloned, sequenced and confer increased UV resistance when introduced into UV-sensitive strains of E. coli, S. cerevisiae, and human cells (Yajima et al. 1995. supra; Takao et al. 1996. supra). In S. pombe Uvde1 is encoded by the uve1+ gene. However, because of the apparently unstable nature of partially purified full-length and some truncated Uvde1 derivatives, Uvde1 enzymes have been relatively poorly characterized and are of limited use (Takao et al. 1996. supra).

Because of the increasing and widespread incidence of skin cancers throughout the world and due to the reported inherent instability of various types of partially purified full-length and truncated Uvde1 derivatives, there is a long felt need for the isolation and purification of stable Uvde1 products, especially for use in skin care and medicinal formulations.

### BRIEF SUMMARY OF THE INVENTION

The present invention provides a re-engineered ultraviolet (UV) damage endonuclease (Uve1p, UVE, Uvde1), termed Pombe-Pro herein. It comprises a catalytic region composed of amino acids 96-503 of the full length (natural) Uvde1 protein. See Table 2 for the His-tagged truncated protein.

A further aspect of the present invention is a Δ95 derivative of Uvde1 (lacking amino acids 1-95 of the naturally occurring protein) further comprising a cell-penetrating domain, which as specifically exemplified is a Tat peptide, and optionally, to facilitate purification after recombinant production, a tag peptide specifically exemplified by a well known hexahistidine portion. As specifically exemplified, the Tat peptide fused with the Δ95 derivative has the amino acid sequence YGRKKRRQRRR (SEQ ID NO1), which corresponds to amino acids 47-57 of the Tat protein. The complete protein sequence is given in Table 2, and a specifically exemplified coding sequence is given in Table 3. Other cell penetrating domains can be substituted for the Tat-derived peptide portion, including but not limited to amino acids 48-60 (RKKRRQRRRAHQ, SEQ ID NO:2) of the HIV type 1 Tat protein (Vives et al. 1997. J. Biol. Chem. 272(25):16010-16017), amino acids 43-58 of the Antennapedia protein (RQRIKIWFQNRRMKWKK, SEQ ID NO:3) from Drosophila (Christiaens et al. 2004. Eur. J. Biochem. 271:1187-1197; Derossi et al. 1996. J. Biol. Chem. 271(30):18188-18193), amino acids 35 to 47 of the human Clock protein (RVSRNKSEKKRR, SEQ ID NO:4), amino acids 831-845 of hPer1 (RRHHCRSKAKRSRHH, SEQ ID NO:5) and amino acids 789-806 of hPer2 (KKTGKNRKLKSKRVKPRD, SEQ ID NO:6) (Peng et al. 2004. Acta. Biochim. Biophys. Sinica 36:629-636), as well peptides derived in sequence from HIC Rev, flock house virus coat proteins, DNA binding segments of leucine zipper proteins including the cancer-related proteins c-Fos and c-Jun, and the yeast transcription factor GCN4. The sequence RKKRRQRRR (SEQ ID NO:7, derived from the HIV Tat protein) can also be incorporated in Uvde1 protein fusions which are efficiently transported into cells via the Tat-derived sequence. An additional cell penetration peptide (KETWWETWWTEWSQPKKKRKV, SEQ ID NO:8) is described in Morris et al. 2001. Nat. Biotechnol. 19:1173-1176. While these cell penetration peptides can be incorporated as part of a Uvde1-derived fusion protein by molecular biological methods, these peptides can also be mixed with the Uvde1-derived protein of the present invention in compositions for topical application or for parenteral administration, and the peptides will complex with the Uvde1-derived protein and mediate transfer into cells where the beneficial DNA repair activity takes place.

Other tag peptides which can be engineered into the primary structure of the truncated Uvde1 useful to facilitate purification of the present re-engineered Uvde1 include the streptavidin (biotin-binding) tag, a flagellar antigen tag, a hemagglutinin tag, a glutathionine S-transferase tag, or a polyhistidine tag, among others.

Also within the scope of the present invention are recombinant DNA molecules encoding the re-engineered Uvde1 of the present invention. A specifically exemplified coding sequence is given in Tables 1 and 3, but all synonymous coding sequences are encompassed within the invention.

An additional aspect of the present invention is methods for reducing aging of skin due to exposure to UV, for example, due to tanning and/or sunburn, or due to exposure of to a mutagenic compound such as a DNA intercalating agent, a DNA methylating agent, including occupational exposure. The methods encompass the step of applying a composition comprising as the active ingredient an amount of the re-engineered Uvde1 of the present invention together with a cell penetration peptide (either as a separate molecule or as part of a fusion protein construct), and a carrier acceptable and useful for topical application of a protein. The Uvde1-Δ95 protein, together with a cell penetration peptide, can be incorporated in a lotion, potion, cream, ointment or other formulation suitable for topical administration, or it can be incorporated in a pharmaceutical composition suitable for parenteral administration, for example, intravenous administration.

Also within the scope of the present invention are nucleic acid molecules encoding such polypeptide fragments and recombinant cells, tissues and animals containing such nucleic acids or polypeptide fragments, antibodies to the polypeptide fragments, assays utilizing the polypeptide fragments, pharmaceutical and/or cosmetic preparations containing the polypeptide fragments and methods relating to all of the foregoing.

A specifically exemplified embodiment of the invention is an isolated, enriched, or purified nucleic acid molecule encoding Uvde1-Δ95. Another exemplified embodiment is an isolated, enriched or purified nucleic acid molecule encoding Tat-Uvde1-Δ95.

In a specifically exemplified embodiment, the isolated nucleic acid comprises, consists essentially of, or consists of a nucleic acid sequence set forth in Table 3.

In another embodiment, the invention encompasses a recombinant cell containing a nucleic acid molecule encoding Uvde1-Δ95 or Tat-Uvde1-Δ95. The recombinant nucleic acid may contain a sequence set forth in Table 3, a synonymous coding sequence or a functional derivative. In such cells, the Uvde1-Δ95 coding sequence is generally expressed under the control of heterologous regulatory elements including a heterologous promoter that is not normally coupled transcriptionally to the coding sequence for the Uvde1 polypeptide in its native state.

In yet another aspect, the invention relates to a nucleic acid vector comprising a nucleotide sequence encoding Uvde1-Δ95, Tat-Uvde1-Δ95 or Tat-Uvde1-Δ95-His-tag and transcription and translation control sequences effective to initiate transcription and subsequent protein synthesis in a host cell. Where a His-tagged Uvde1 derivative is expressed, the His tag portion is desirably removed (after affinity purification) by protease cleavage, for example using thrombin, but the presence of the His tag does not negatively impact activity, cell penetration (in the case of Tat or other cell penetration peptide fusion proteins) or stability during storage.

The present invention further provides methods for cleaving DNA molecules at positions with structural distortions, wherein the DNA is cleaved in the vicinity of the distortion by a stable truncated Uvde1 protein of the present invention. The structural distortion can result from mismatch at the site of the distortion in a double-stranded DNA molecule, from UV damage or from other damage to DNA due to chemical reaction, for example, with an alkylating or depurination agent or due to damage due to UV irradiation, ionizing radiation or other irradiation damage. Abasic sites, mismatch, intercalated molecules such as ethidium bromide, and adducts formed by cisplatin compounds also cause distortion of double-stranded DNA and may result in or be the result of mutations; these structural aberrations can also trigger cleavage and repair via the Uvde1 derivative of the present invention. Cumulative damage to DNA can result in an unattractive and aged appearance of the skin, especially on the face, neck and chest, if that damage is not repaired, The stable truncated Uvde1 proteins can be supplied to the skin in substantially pure form for in vitro reactions, or they can be supplied for in vivo reactions, including but not limited to compositions for topical application (in the form or of an ointment, salve, cream, lotion, liquid or transdermal patch) in compositions for topical administration, with the result that damage to skin cells is reduced and the apparent aging of the skin is reduced. The Uvde1 derivatives of the present invention can also be used to treat potential systemic DNA damage via internal use (to be administered by intraperitoneal, intradermal, subcutaneous, intravenous or intramuscular injection), for example, after exposure to a mutagenic compound to which a person has been exposed, with the result that systemic damage resulting from distorted DNA structure and mutagenesis is reduced. The stable truncated Uvde1 derivatives of the present invention repair a wide variety of mismatch and DNA damage. The cleavage of a double stranded DNA molecule having structural distortion due to nucleotide mispairing (mismatch) or due to DNA damage by a stable truncated Uvde1 derivative of the present invention can be used to advantage in a relatively simple assay for structural distortion wherein cleavage of a test molecule (i.e., the double stranded DNA molecule which is being screened for damage, mismatch or other structural distortion) is to be detected.

The present invention further provides a method for cleaving a double stranded DNA molecule in which there is a structural distortion. The structural distortion can be due to aberrations including, but not limited to, base pair mismatch, photoproduct formation, alkylation of a nucleic acid molecule such that normal Watson-Crick base pairing is disturbed, intercalation between nucleotides of a compound which could be, for example, an acriflavine, an ethidium halide, among others, or a platinum adduct, for example of a cisplatin moiety. The distortion can also be due to an insertion-deletion loop of five or fewer nucleotides in one of the two strands. Desirably, the loop has four or fewer nucleotides. The DNA can also contain an abasic site, a uracil residue resulting from deamination of a cytosine residue, among others. The method of the present invention can be employed using the truncated Uvde1 (UVDE) protein from Schizosaccharomyces pombe, a truncated derivative of the S. pombe Uvde1 (lacking from 90-100 N-terminal amino acids, desirably 95, as specifically exemplified by the Uvde1-Δ95 of S. pombe, or a tagged Uvde1-Δ95. Specifically exemplified truncated Uvde1-Δ95 is given in Table 2. DNA containing the structural distortion is contacted with an enzyme (or active truncated derivative) as described above under conditions allowing endonucleolytic cleavage of one strand of the distorted DNA molecule.

### BRIEF DESCRIPTION OF THE DRAWINGS

FIG. 1A presents a schematic for Uvde1 re-engineering into Pombe-Pro, and FIGS. 1B-1E document its apparent molecular weight, activity and stability. FIG. 1A: Comparison of unmodified (top) Uvde1, N-terminal truncated Uvde1-Δ95 (middle) and tagged, truncated Pombe-Pro (Tat-Uvde1-Δ95) (bottom). Not shown is the C-terminal His₆ tag attached to the C-terminus to achieve rapid, efficient purification. FIG. 1B: SDS-PAGE analysis of purified Pombe-Pro. Lane 1: protein size markers; lane 2: 0.5 μg Pombe-Pro; lane 3: 3 μg Pombe-Pro. FIG. 1C: DNA repair (cleavage) activity of Pombe-Pro (0.6 μg) using A/C mismatch damage-containing oligonucleotide as substrate. FIG. 1D: SDS-PAGE analysis of 2 μg purified Pombe-Pro following storage in solution at 4° C. for one month. FIG. 1E: DNA repair (cleavage) activity of Pombe-Pro using A/C mismatch damage-containing oligonucleotide of indicated amounts of Pombe-Pro shown in FIG. 1D.

FIGS. 2A-C illustrate the immunocytochemical analysis of human keratinocytes (HaCat cells) transfected with Pombe-Pro. Cells were exposed to Pombe-Pro (12 micrograms) for 30 minutes before fixing and staining. The presence of Pombe-Pro in cells was revealed by treatment with Uvde-1 antibody (polyclonal), followed by treatment with an FITC-conjugated secondary antibody. FIG. 2A: FITC image (fluorescence microscopy) revealing the intracellular presence of Pombe-Pro. FIG. 2B: DAPI-stained cells indicating nuclear DNA. FIG. 2C: Merged images of FIGS. 2A and 2B demonstrating Pombe-Pro localization to nucleus.

FIG. 3 demonstrates that Pombe-Pro has no effect on human keratinocyte cell growth. Human keratinocyte cultures were exposed to a biologically active dose (3 micrograms) of Pombe-Pro and the potential effect on cell growth over the time period indicated was compared to unexposed cells.

FIGS. 4A-4B show that Pombe-Pro (Tat-Uvde1) reduces levels of UV-induced DNA damage in human keratinocytes. FIG. 4A: Comet assay in which DNA damages were assessed 1 hour following exposure of cells to a moderately cytotoxic dose (10 J/m2) of UV light. FIG. 4B: Immunocytochemical analysis employing CPD monoclonal antibody. CPD levels were assessed 3 hours following exposure to 20 J/m2 of UV light.

FIGS. 5A-5B show that Pombe-Pro (Tat-Uvde1-Δ95-his) increases human keratinocyte cell survival following exposure to UV radiation. FIG. 5A: Cell survival following UV exposure at indicated doses and treatment with (closed bars) or without (open bars) treatment with Pombe-Pro (3 micrograms). FIG. 5B: Relative cell survival following UV exposure (10 J/m2) and treatment with the indicated proteins (3 micrograms).

### DETAILED DESCRIPTION OF THE INVENTION

Sunlight exposure-associated human skin cancer is a major public health problem. In addition, skin photoaging from daily sunlight exposure is a major concern in the cosmetics industry. The reengineered version of the DNA-repair protein Uvde1 from Schizosaccharomyces pombe (African beer yeast) described herein reduces DNA damage caused by UV radiation and consequently increases human skin cell survival to UV exposure. Accordingly, this natural agent is advantageously used for the reduction in the extent or risk of and/or prevention of human skin cancer and skin photoaging caused by sunlight exposure. The engineered proteins of the present invention can be formulated into cosmeceutical as well as pharmaceutical products. The engineered Uvde1 derivative recognizes and initiates the repair of the two major cytotoxic, mutagenic and carcinogenic types of DNA damage caused by exposure of cells to UV irradiation. In addition, this protein recognizes other types of DNA damage, including some types of DNA damage caused by oxygen radicals and distortions of double stranded DNA due to mismatch, abasic sites and short insertion/deletion loops.

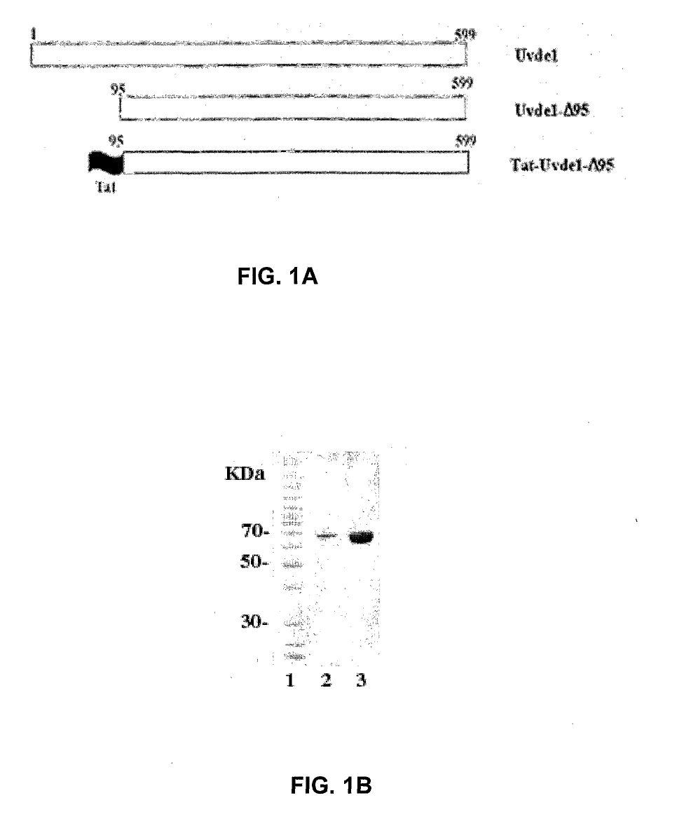

A large quantity of highly purified, biochemically modified, active (re-engineered) Uvde1 protein enabled the present study. Despite technical problems with Uvde1 and its derivatives encountered earlier (certain forms of Uvde1 are unstable and possess low solubility), we purified large amounts of the re-engineered version of Uvde1 (Uvde1-Δ95), an N-terminal truncated version of Uvde1, after expression in Escherichia coli BL21 (DE3). To facilitate delivery of purified Pombe-Pro into human skin cells, we fused the Tat cell penetration peptide to the N-terminus (FIGS. 1A-1C). To facilitate rapid, simple purification of Uvde1-Δ95 and its derivatives, we also attached a His6 (six histidine residues) tag to the C-terminus of the protein. We have demonstrated that both Uvde1-Δ95 and the His-tagged Tat fusion protein Pombe-Pro are enzymatically active (FIG. 1C). Furthermore, we have demonstrated that both Uvde1-Δ95 and Pombe-Pro are relatively stable and retain activity for months under proper storage conditions (−20° C.). Even with storage for one month under standard refrigeration temperatures (4° C.), Pombe-Pro (in solution) retains greater than 60% of its original activity (FIGS. 1D and 1E).

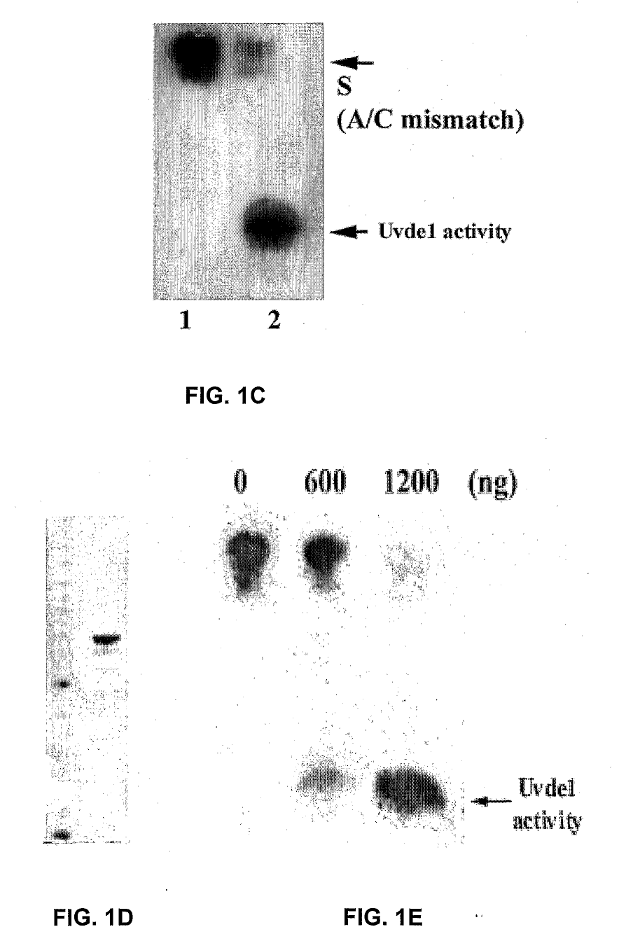

An important aspect of the present invention is directing the Uvde1-derived protein into cells where it can exert its beneficial effect on DNA distorted by damage such as that from ultraviolet irradiation or other deleterious agents that cause distortions within double-stranded DNA. The fusion of the Tat peptide tag with Uvde1-Δ95 allowed us to directly deliver protein into human skin keratinocytes (HaCat cells). Keratinocytes are useful for proof-of-principle studies because they represent the precursor cells that can develop into basal and squamous cell carcinomas, the most frequently occurring human skin cancers caused by chronic exposure to sunlight. Optimal conditions (incubation of cells with protein in PBS buffer for 30 minutes at room temperature) for protein delivery into human skin cells was established and verified via immunocytochemistry (FIGS. 2A-2C) and western blot analysis (not shown).

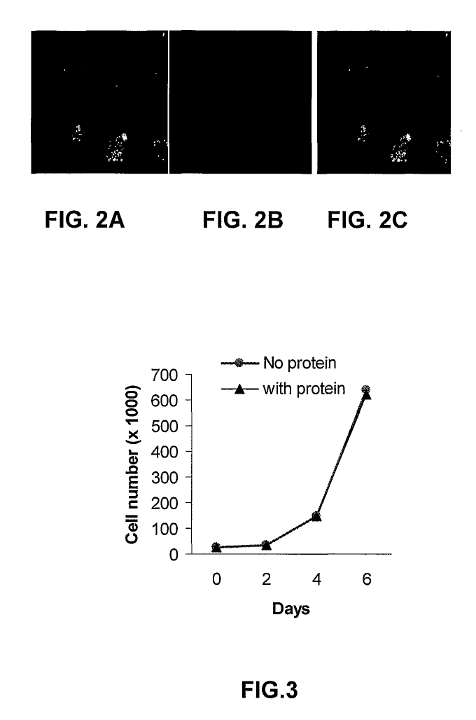

The effect of Pombe-Pro on HaCat cell growth was determined in assess the potential cytotoxic or cytostatic effects mediated by this protein. Exposure to doses of Pombe-Pro that result in protection from UV light-induced DNA damage and cytotoxicity did not result in any measurable effects on cell growth or viability. We have concluded that at the doses tested, Pombe-Pro is non-toxic (see FIG. 3).

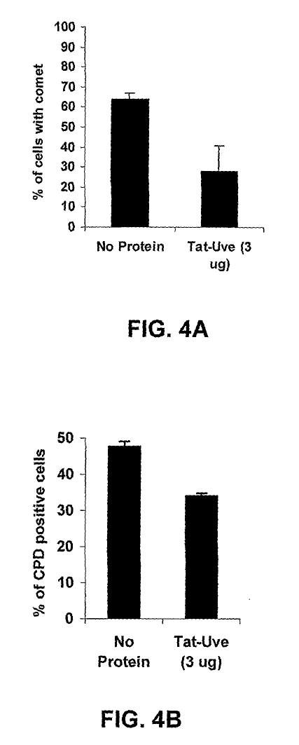

Two types of studies were conducted to examine whether Pombe-Pro reduces cellular DNA damage levels following exposure to UV radiation. First, comet assays, which are based on the principle that nuclear DNA containing unpaired damages are fragmented following UV irradiation and alkali exposure causing broken DNA fragments to migrate out of the nucleus to form a “comet” following electrophoresis of the cell/DNA slide preparation were performed. Such comets are visualized and quantified via microscopic analysis of stained DNA. Thus, it is an assay to determine the overall level of cellular DNA damage level. UV-irradiated human keratinocytes exposed to non-toxic doses of Tat-Uvde1-Δ95 demonstrated a significant reduction of DNA damage-induced comets, indicating that Pombe-Pro can initiate the repair of UV damage in vivo (FIG. 4A). This important observation has been verified using an immunocytochemical assay employing a monoclonal antibody against cyclobutane pyrimidine dimers (CPDs) to directly assess the levels of CPDs (a major mutagenic and carcinogenic UV light-induced DNA damage product) in human keratinocytes, following exposure to UV radiation. Our results demonstrate that Pombe-Pro reduced the overall CPD levels in cells by about 15-20% (FIG. 4B). We conclude from these results that Pombe-Pro is capable of initiating the repair and elimination of cytotoxic, mutagenic, carcinogenic DNA damage caused by UV light in human keratinocytes.

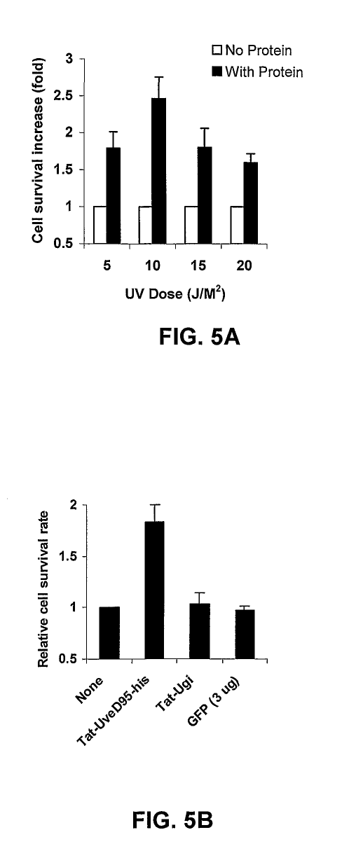

We have also utilized a colony formation assay to further examine whether Pombe-Pro increases cell survival after UV exposure. Multiple experiments utilizing this assay under different UV exposure conditions demonstrate that Pombe-Pro increases human keratinocyte (HaCat cells) survival following exposure to UV radiation (FIG. 5A). The average increase of cell survival is about 2-fold (representing a 90-100% increase). Importantly, we have demonstrated that the increase of cell survival conferred by Pombe-Pro is specific for this DNA repair enzyme, since incubation of other proteins such as GFP (green fluorescent protein) or Tat-Ugi (Tat-tagged protein inhibitor of uracil glycosylase) following UV exposure was without effect (FIG. 5B).

We have demonstrated within the context of a human skin keratinocyte cell culture system that the re-engineered yeast DNA repair protein, Pombe-Pro, penetrates cells and localizes to the nucleus upon exposure of cells to purified protein; that it initiates the repair of UV radiation-induced cytotoxic, mutagenic and carcinogenic DNA damage; and that it increases the survival of UV-damaged cells at non-toxic doses. The degree of protection afforded by Pombe-Pro is significant biologically as even small increases in the overall DNA repair capacity of cells translates into a major, additional protective effect against tumor development. Similarly, the beneficial effects of Pombe-Pro or a derivative thereof retard the aging of skin cells, especially in the context of UV exposure and/or exposure to certain mutagenic agents or conditions that otherwise would result in DNA damage characterized by distortion of the double-stranded DNA.

Based on the data provided herein, we have concluded that Pombe-Pro enters skin cells and that it, when administered in a therapeutically effective dose, protects against UV light-induced skin cancer development and photoaging. This is verified in an appropriate system, such as the SKH1 hairless mouse, an accepted animal model for UV photocarcinogenesis studies.

The full length S. pombe Uvde1 protein and its coding sequence are given in Table 1. The sequence of the Uvde1-Δ95 corresponds to amino acids 96-503 of the amino acid sequence given in Table 1. The coding and amino acid sequences for the His-tagged, Tat-fused Uvde1-Δ95 protein is given in Table 3. A variety of recombinant host cells can be used to produce the protein of the present invention including, but not limited to, mammalian cells such as CHO cells or Vero cells, yeast cells such as Pichia pastoris or Saccharomyces cerevisiae, fungal cells such as Trichoderma reesei or Aspergillus nidulans, or bacterial cells including Escherichia coli, Bacillus subtilis, Pseudomonas fluorescens, or others.

The re-engineered Uvde1 proteins of the present invention can be readily engineered to facilitate purification and/or immobilization to a solid support of choice. For example, a stretch of 5-8 histidines can be engineered through mutagenic polymerase chain reaction, through the use of available cloning vectors or other recombinant DNA technology to allow or facilitate purification of expressed recombinant protein over a nitrilotriacetic acid (NTA) affinity column using commercially available materials, for example, Ni-NTZ Agarose from Qiagen, Valencia, Calif. As specifically exemplified, the His tag is fused to the C-terminus of the Uvde1-Δ95 protein to facilitate purification, and if desired, the Tat or other cell penetrating peptide portion is engineered to the N-terminus of the protein. This is accomplished by site-directed mutagenesis/recombinant DNA technology. Oligopeptide “tags” which can be fused to a protein of interest by such techniques include, without limitation, strep-tag (Sigma-Genosys, The Woodlands, Tex.) which directs binding to streptavidin or its derivative streptactin (Sigma-Genosys); a glutathione-S-transferase gene fusion system which directs binding to glutathione coupled to a solid support (Amersham Pharmacia Biotech, Uppsala, Sweden); a calmodulin-binding peptide fusion system which allows purification using a calmodulin resin (Stratagene, La Jolla, Calif.); a maltose binding protein fusion system allowing binding to an amylose resin (New England Biolabs, Beverly, Mass.); and an oligo-histidine fusion peptide system which allows purification using a Ni²⁺-NTA column (Qiagen, Valencia, Calif.).

In general the terms and phrases used herein have their art-recognized meaning, which can be found by reference to standard texts, journal references and contexts known to those skilled in the art. The following definitions are provided to clarify their specific use in the context of the invention.

A cell penetration peptide is one which has a membrane permeability and carrier function for intracellular protein delivery. Typically, such a protein has an overall net positive charge, and is likely to be rich in arginine and/or lysine. A cell penetration peptide useful in the present invention may include D-amino acids instead of or in addition to L-amino acids. Specific examples of cell penetration peptides include, but are not limited to, those corresponding in amino acid sequence to amino acids 47 to 57 (YGRKKRRQRRR, SEQ ID NO:1) and amino acids 48-60 (RKKRRQRRAHQ, SEQ ID NO:2) of the HIV type 1 Tat protein (Vives et al. 1997. J. Biol. Chem. 272(25):16010-16017), amino acids 43-58 of the Antennapedia protein (RQRIKIWFQNRRMKWKK, SEQ ID NO:3) from Drosophila (Christiaens et al. 2004. Eur. J. Biochem. 271:1187-1197; Derossi et al. 1996. J. Biol. Chem. 271(30):18188-18193), amino acids 35 to 47 of the human Clock protein (RVSRNKSEKKRR, SEQ ID NO:4), amino acids 831-845 of hPer1 (RRHHCRSKAKSRHH, SEQ ID NO:5) and amino acids 789-806 of hPer2 (KKTGKNRKLKSKRVKPRD, SEQ ID NO:6) (Peng et al. 2004. Acta. Biochim. Biophys. Sinica 36:629-636), as well as peptides derived in sequence from HIC Rev, flock house virus coat proteins, DNA binding segments of leucine zipper proteins including the cancer-related proteins c-Fos and c-Jun, and the yeast transcription factor GCN4. US Patent Publication No. 2003/0185862 describes fusion proteins containing the sequence RKKRRQRRR (SEQ ID NO:7 derived from the HIV Tat protein) which are efficiently transported into cells via the Tat-derived sequence. An additional cell penetration peptide (KETWWETWWTEWSQPKKKRKV, SEQ ID NO:8) is described in Morris et al. 2001. Nat. Biotechnol. 19:1173-1176. See also Schwarze et al. 1999. Science 285:1569-1572; Futaki et al. 2001. J. Biol. Chem. 276:5836-5840; Schwarze et al. 2000. Trends Pharmacol. Sci. 21:45-48, for a discussion of peptide-mediated intracellular transport.

Every formulation or combination of components described or exemplified can be used to practice the invention, unless otherwise stated. Specific names of compounds are intended to be exemplary, as it is known that one of ordinary skill in the art can name the same compounds differently. When a compound is described herein such that a particular isomer or enantiomer of the compound is not specified, for example, in a formula or in a chemical name, that description is intended to include each isomers and enantiomer of the compound described individual or in any combination. One of ordinary skill in the art will appreciate that methods, starting materials, synthetic methods, protein purification methods and molecular biological methods other than those specifically exemplified can be employed in the practice of the invention without resort to undue experimentation. All art-known functional equivalents, of any such methods, device elements, starting materials, synthetic methods, purification methods and molecular biological techniques are intended to be included in this invention. Whenever a range is given in the specification, for example, a temperature range, a time range, or a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be carried out by those skilled in the art, and that such modifications and variations are considered to be within the scope of this invention as defined by the appended claims.

In general the terms and phrases used herein have their art-recognized meaning, which can be found by reference to standard texts, journal references and contexts known to those skilled in the art. The following definitions are provided to clarify their specific use in the context of the invention.

The exact formulation, route of administration and dosage can be chosen by the individual physician in view of the patient's condition (see e.g. Fingl et al., in The Pharmacological Basis of Therapeutics, 1975, Ch. 1 p. 1).

It should be noted that the attending physician knows how to and when to terminate, interrupt, or adjust administration due to toxicity, or to organ dysfunctions, or to immunological reactions. Conversely, the attending physician also knows to adjust treatment to higher levels if the clinical response were not adequate (precluding toxicity or other adverse reaction). The magnitude of an administered dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above also may be used in veterinary medicine.

Depending on the specific conditions being treated and the targeting method selected, such agents may be formulated and administered systemically or locally. Techniques for formulation and administration are well known to the art. Suitable routes may include, for example, topical, transdermal, transmucosal, or intestinal administration; or by parenteral delivery, including intramuscular, subcutaneous, or intramedullary injections, as well as intrathecal, intravenous, or intraperitoneal injections. For reducing the damaging effects of ultraviolet and/or sun exposure of the skin, topical application is preferred.

For injection, the agents of the invention may be formulated in aqueous solutions, preferably in physiologically compatible buffers such as Hanks' solution, Ringer's solution, or physiological saline buffer. For topical or transmucosal administration, penetrants appropriate to the barrier to be permeated are used in the formulation. Such topical formulations and penetrants are generally known in the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular those formulated as solutions, may be administered parenterally, such as by intravenous injection. Appropriate compounds can be formulated readily using pharmaceutically acceptable carriers well known in the art into dosages suitable for oral administration. Such carriers enable the compounds of the invention to be formulated as lotions, creams, ointments, transdermal patches, or into tablets, pills, capsules, liquids, gels, syrups, slurries, suspensions and the like, for oral ingestion by a patient to be treated.

Delivery of the re-engineered Uvde1 or Uvde1-Δ95 protein (to be administered and transported intracellularly) can be accomplished using techniques well known to those of ordinary skill in the art. For example, as an alternative or in addition to the cell penetration peptides discussed herein above, the Uvde1 or Uvde1-Δ95 protein may be encapsulated into liposomes, then administered topically in a dosage sufficient to reduce DNA damage resulting from exposure to UV light, sunlight or mutagenic compounds that cause distortion of double stranded DNA. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to their hydrophobicity, small organic molecules may be directly administered intracellularly.

Pharmaceutical compositions suitable for use in the present invention include compositions wherein the active ingredients are contained in an effective amount to achieve the intended purpose. Determination of the effective amounts is well within the capability of those skilled in the art, especially in light of the detailed disclosure provided herein. For example, the Uvde1-Δ95 can be incorporated into a topical medicament or cosmeceutical such that a dose of 0.01 to 10 mg per 100 grams of medicament can be delivered. Alternatively, the Uvde1-Δ95 or derivative can be incorporated into a liposomal formulation of from 0.1 to 10 mg/L, from about 0.5 to about 3, or about 1 mg/L and applied to the face in a volume of about 3 to 6, or about 4 to 5 mL per day. Topical application to other body parts exposed to the sun, especially soon after sunburn or overexposure, can be accomplished at similar rates per unit area. Where the Uvde1-Δ95 is to be administered systemically, a dose of from 0.1 to 10 mg per kg body weight can be used to ameliorate damage and/or mutation resulting from a source of DNA distortion, including by not limited to alkylation, intercalation, deamination, mismatch, insertion or deletions of from 1 to 4 or 5 nucleotides, among others. See U.S. Pat. No. 6,368,594 for a discussion of the range of DNA distortions recognized by the Uvde1 enzyme.

In addition to the active ingredients, these pharmaceutical compositions may contain suitable pharmaceutically acceptable carriers comprising excipients and auxiliaries which facilitate processing of the active compounds into preparations which can be used pharmaceutically. For topical use, formulations suited by cosmetic (or cosmeceutical) use are particularly useful. Coloring and/or fragrance and emollients can be incorporated into the formulation for topical application, as known to the art, to improve the feeling of well being by the user.

The pharmaceutical compositions of the present invention may be manufactured in a manner that is itself known, e.g., by means of conventional mixing, dissolving, granulating, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

All art-known functional equivalents of methods, starting materials, synthetic methods, pharmaceutical formulations and delivery methods are intended to be included in the present invention. Whenever a range is given in the specification, for example, a temperature range, a time range, or a composition range, all intermediate ranges and subranges, as well as all individual values included in the ranges given are specifically included within the scope of the present invention.

Agents intended to be administered intracellularly may be administered using techniques well known to those of ordinary skill in the art. For example, such agents may be encapsulated into liposomes, then administered as described above. Liposomes are spherical particles with aqueous interiors bounded lipid bilayers. Soluble molecules present in an aqueous solution at the time of liposome formation are incorporated into the aqueous interior. The liposomal contents are both protected from the external microenvironment and, because liposomes fuse with cell membranes, the contents are efficiently delivered into the cell cytoplasm. Additionally, due to their hydrophobicity, small organic molecules may be directly administered intracellularly.

Pharmaceutical or cosmeceutical compositions suitable for use in the present invention include compositions wherein the Uvde1-Δ95 or derivative thereof is present in an effective amount to achieve the intended purpose of decreasing damage to DNA. For topical application of such cosmeceutical compositions after exposure to ultraviolet light, for example from sunshine, reduces UV-induced damage to the skin and promotes the retention of a youthful appearance. Determination of the effective amount is well within the capability of those skilled in the art.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Monoclonal or polyclonal antibodies, preferably monoclonal, specifically reacting with a protein or peptide of interest may be made by methods known in the art. See, e.g., Harlow and Lane (1988) Antibodies: A Laboratory Manual, Cold Spring Harbor Laboratories; Goding (1986) Monoclonal Antibodies: Principles and Practice, 2d ed., Academic Press, New York.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; and Setlow and Hollaender (1979) Genetic Engineering: Principles and Methods, Vols. 1-4, Plenum Press, New York. Abbreviations and nomenclature, where employed, are deemed standard in the field and commonly used in professional journals such as those cited herein.

All references cited herein are hereby incorporated by reference to the extent there is no inconsistency with the present disclosure. The references cited herein reflect of skill in the relevant art.

Although the description herein contains certain specific descriptions, examples and embodiments, these should not be construed as limiting the scope of the invention but rather as providing illustrations of some of the presently preferred embodiments of the invention. For example, thus the scope of the invention should be determined by the appended claims and their equivalents, rather than by the examples given.

### Stability of Protein

The stability of Tat-Uvde1-Δ95-his has been tested. Stored at 4° C., the protein is fairly stable. After one month, about 60% of protein remains intact and biochemically active. However, the protein is not stable at room temperature (25° C.), with protein degradation beginning during the first 24 hours. By day three, half of protein is intact; and by day 7, all protein is gone. The degradation of protein could be due to contamination of proteinase(s) during protein purification. Therefore, further purification or addition of proteinase inhibitors to the protein preparation can increase the stability of this protein. Such proteinase inhibitors can include, without limitation, phenyl methylsulfonyl fluoride, leupeptin, soybean trypsin inhibitor, pepstatin A, EDTA, 4(2-aminoethyl)benzenesulfonyl fluoride, E-64, phosphoramidon, antipain, aprotin, chymostatin, 1,10-phenanthroline, bestatin, soybean trypsin inhibitor, and others.

### Protein Purification

The standard protein purification procedure includes a high performance cation exchange chromatography step (e.g., SP Sepharose column, GE Healthcare, with 20 mM KH₂PO₄, pH 7.4, 0.5 mM EDTA, in 10% glycerol, eluted with a 0.1 to 1.0 M KCL gradient), followed by Nickel affinity purification and the second round of high performance cation exchange chromatography. Interestingly, addition of Tat to protein appears to increase the binding ability of the engineered protein to the SP column. While Uvde1-Δ95-His is eluted from SP with about 350 mM KCl, Tat-Uvde1-Δ95-His is eluted from SP column at a higher salt concentration (about 1 M KCL). This facilitates purification of Tat-Uvde1-Δ95-his because most contaminating proteins are washed away with 600 mM KCl.

TABLE 1
Uvde1-Δ95-His Coding Sequence (SEQ ID NO:9)

ATGggacctc acaaaaaaag tacttctacg tctacacgaa agagggcacg
tagcagtaaa aagaaagcga cagattctgt ttccgataaa attgatgagt ctgttgcgtc
ctatgattct tcaactcatc ttaggcgatc gtcgagatca aaaaaaccgg tcaactacaa
ttcctcgtca gaatccgaat cggaggagca aattagtaaa gctactaaaa aagttaaaca
aaaagaggaa gaggagtatg ttgaagaagt cgacgaaaag tctcttaaaa atgaaagtag
ctctgacgag ttcgaaccgg ttgtgccgga acagttggaa actccaattt ctaaacgaag
acggtctcgt tcttctgcaa aaaatttaga aaaagaatct acaatgaatc ttgatgatca
tgctccacga gagatgtttg attgtttgga caaacccata ccctggcgag gacgattggg
gtatgcttgt ttgaatacta ttttaaggtc aatgaaggag agggtttttt gttcacgcac
ctgccgaatt acaaccattc aacgtgatgg gctcgaaagt gtcaagcagc taggtacgca
aaatgtttta gatttaatca aattggttga gtggaatcac aactttggca ttcacttcat
gagagtgagt tctgatttat ttcctttcgc aagccatgca aagtatggat atacccttga
atttgcacaa tctcatctcg aggaggtggg caagctggca aataaatata atcatcgatt
gactatgcat cctggtcagt acacccagat agcctctcca cgagaagtcg tagttgattc
ggcaatacgt gatttggctt atcatgatga aattctcagt cgtatgaagt tgaatgaaca
attaaataaa gacgctgttt taattattca ccttggtggt acctttgaag gaaaaaaaga
aacattggat aggtttcgta aaaattatca acgcttgtct gattcggtta aagctcgttt
agttttagaa aacgatgatg tttcttggtc agttcaagat ttattacctt tatgccaaga
acttaatatt cctctagttt tggattggca tcatcacaac atagtgccag gaacgcttcg
tgaaggaagt ttagatttaa tgccattaat cccaactatt cgagaaacct ggacaagaaa
gggaattaca cagaagcaac attactcaga atcggctgat ccaacggcga tttctgggat
gaaacgacgt gctcactctg atagggtgtt tgactttcca ccgtgtgatc ctacaatgga
tctaatgata gaagctaagg aaaaggaaca ggctgtattt gaattgtgta gacgttatga
gttacaaaat ccaccatgtc ctcttgaaat tatggggcct gaatacgatc aaactcgaga
tggatattat ccgcccggag ctgaaaagcg tttaactgca agaaaaaggc gtagtagaaa
agaagaagta gaagaggatg aaaaa CAT CAC CAT CAC CAT CAC taa

Note:
Underlined residues are not part of endogenous sequences of Uvde1.
They are added by engineering design.

TABLE 2
Uvde1-Δ95-His Protein Sequence (SEQ ID NO:10)

Mgphkk stststrkra rsskkkatds vsdkidesva sydssthlrr ssrskkpvny nsssesesee
qiskatkkvk qkeeeeyvee vdekslknes ssdefepvvp eqletpiskr rrsrssaknl ekestmnldd
hapremfdcl dkpipwrgrl gyaclntilr smkervfcsr tcrittiqrd glesvkqlgt qnvldliklv
ewnhnfgihf mrvssdlfpf ashakygytl efaqshleev gklankynhr ltmhpgqytq
iasprevvvd sairdlayhd eilsrmklne qlnkdavlii hlggtfegkk etldrfrkny qrlsdsvkar
lvlenddvsw svqdllplcq elniplvldw hhhnivpgtl regsldlmpl iptiretwtr kgitqkqhys
esadptaisg mkrrahsdrv fdfppcdptm dlmieakeke qavfelcrry elqnppcple imgpeydqtr
dgyyppgaek rltarkrrsr keeveedeKH HHHHH

Note:
Underlined residues are not part of endogenous sequences of Uvde1.
They are added by engineering design.

TABLE 3
TAT-Uvde1-Δ95-His Coding Sequence (SEQ ID NO:11)

ATG GGA TAC GGC CGC AAG AAA CGC CGC CAG CGC CGA CGC GGT
ggacctc acaaaaaaag tacttctacg tctacacgaa agagggcacg
tagcagtaaa aagaaagcga cagattctgt ttccgataaa attgatgagt ctgttgcgtc
ctatgattct tcaactcatc ttaggcgatc gtcgagatca aaaaaaccgg tcaactacaa
ttcctcgtca gaatccgaat cggaggagca aattagtaaa gctactaaaa aagttaaaca
aaaagaggaa gaggagtatg ttgaagaagt cgacgaaaag tctcttaaaa atgaaagtag
ctctgacgag ttcgaaccgg ttgtgccgga acagttggaa actccaattt ctaaacgaag
acggtctcgt tcttctgcaa aaaatttaga aaaagaatct acaatgaatc ttgatgatca
tgctccacga gagatgtttg attgtttgga caaacccata ccctggcgag gacgattggg
gtatgcttgt ttgaatacta ttttaaggtc aatgaaggag agggtttttt gttcacgcac
ctgccgaatt acaaccattc aacgtgatgg gctcgaaagt gtcaagcagc taggtacgca
aaatgtttta gatttaatca aattggttga gtggaatcac aactttggca ttcacttcat
gagagtgagt tctgatttat ttcctttcgc aagccatgca aagtatggat atacccttga
atttgcacaa tctcatctcg aggaggtggg caagctggca aataaatata atcatcgatt
gactatgcat cctggtcagt acacccagat agcctctcca cgagaagtcg tagttgattc
ggcaatacgt gatttggctt atcatgatga aattctcagt cgtatgaagt tgaatgaaca
attaaataaa gacgctgttt taattattca ccttggtggt acctttgaag gaaaaaaaga
aacattggat aggtttcgta aaaattatca acgcttgtct gattcggtta aagctcgttt
agttttagaa aacgatgatg tttcttggtc agttcaagat ttattacctt tatgccaaga
acttaatatt cctctagttt tggattggca tcatcacaac atagtgccag gaacgcttcg
tgaaggaagt ttagatttaa tgccattaat cccaactatt cgagaaacct ggacaagaaa
gggaattaca cagaagcaac attactcaga atcggctgat ccaacggcga tttctgggat
gaaacgacgt gctcactctg atagggtgtt tgactttcca ccgtgtgatc ctacaatgga
tctaatgata gaagctaagg aaaaggaaca ggctgtattt gaattgtgta gacgttatga
gttacaaaat ccaccatgtc ctcttgaaat tatggggcct gaatacgatc aaactcgaga
tggatattat ccgcccggag ctgaaaagcg tttaactgca agaaaaaggc gtagtagaaa
agaagaagta gaagaggatg aaaaa CAT CAC CAT CAC CAT CAC taa

Note:
Underlined residues are not part of endogenous sequences of Uvde1.
They are added by engineering design.

TABLE 4
TAT-Uvdel-A95-His Protein Sequence (SEQ ID NO:12)

MGYGRKKRRQRRRGgphkk stststrkra rsskkkatds
vsdkidesva sydssthlrr ssrskkpvny nsssesesee qiskatkkvk qkeeeeyvee
vdekslknes ssdefepvvp eqletpiskr rrsrssaknl ekestmnldd hapremfdcl
dkpipwrgrl gyaclntilr smkervfcsr tcrittiqrd glesvkqlgt qnvldliklv
ewnhnfgihf mrvssdlfpf ashakygyti efaqshleev gklankynhr ltmhpgqytq
iasprevvvd sairdlayhd eilsrmklne qlnkdavlii hlggtfegkk etldrfrkny
qrisdsvkar lvlenddvsw svqdllplcq elniplvldw hhhnivpgtl regsldlmpl
iptiretwtr kgitqkqhys esadptaisg mkrrahsdrv fdfppcdptm dimieakeke
qavfelcrry elqnppcple imgpeydqtr dgyyppgaek rltarkrrsr keeveedek
HHHHHH

Note:
Underlined sequences are not part of endogenous sequences of Uvde1.
They are added by engineering design.

1. A composition comprising an ultraviolet damage endonuclease consisting essentially of the sequence of SEQ ID NO:10, amino acids 2-505; SEQ ID NO:10, amino acids 1-505; SEQ ID NO:10, amino acids 2-511; SEQ ID NO:10, amino acids 1-511; SEQ ID NO:12, amino acids 1-517; SEQ ID NO:12, or SEQ ID NO:12, amino acids 1-524, a cell penetration peptide and a pharmaceutically acceptable carrier.

2. The composition of claim 1, wherein the pharmaceutically acceptable carrier is suitable for topical administration.

3. The composition of claim 1, wherein the endonuclease is characterized by the amino acid sequence as set forth in amino acids 2-505, 1-505 or 1-511 of SEQ ID NO:10.

4. The composition of claim 1, wherein the pharmaceutically acceptable carrier is suitable for parenteral administration.

5. The composition of claim 1, wherein the cell penetration peptide is expressed as part of a fusion protein also comprising the ultraviolet damage endonuclease.

6. The composition of claim 1, wherein the cell penetration peptide has an amino acid sequence as set forth in any of SEQ ID NOs:1 to 8.

7. The composition of claim 5, wherein the fusion protein consists essentially of the amino acid sequence set forth in amino acids 1-524 or 1-517 of SEQ ID NO:12.

8. The composition of claim 5, wherein the fusion protein is characterized by the amino acid sequence set forth in SEQ ID NO:12.

9. The composition of claim 1 wherein the ultraviolet damage endonuclease further comprises a tag portion which facilitates purification.

10. The composition of claim 9, wherein the tag portion is a polyhistidine tag, a hemagglutinin-derived tag, a flagellar antigen derived tag, a biotin-binding tag or a glutathionine S-transferase-derived tag.

11. A method for reducing sun damage to skin, said method comprising the step of topically administering an effective amount of the composition of claim 2.

12. A truncated Uvde1 protein characterized by a sequence as set forth in SEQ ID NO:10, amino acids 2-505; SEQ ID NO:10, amino acids 1-505; SEQ ID NO:10, amino acids 2-511; SEQ ID NO:10, amino acids 1-511; SEQ ID NO:12, amino acids 1-517; SEQ ID NO:12, or SEQ ID NO:12, amino acids 1-524.

13. The use of a truncated Uvde1 protein according to claim 12 in the formulation of a medicament to reduce skin damage by ultraviolet light or chemical agents which cause DNA distortion.

14. A nucleic acid molecule encoding the truncated Uvde1 protein according to claim 12.

15. The nucleic acid molecule according to claim 14, wherein the truncated Uvde1 protein is encoded by a nucleic acid sequence as set forth in SEQ ID NO:9 or SEQ ID NO:11.

16. A recombinant cell comprising the nucleic acid molecule of claim 14.

17. A method for expressing a truncated Uvde1 protein comprising the step of culturing the recombinant cell according to claim 16 under conditions which allow expression of said protein.


SEQUENCE LISTING (12 sequences)

SEQ ID NO: 1 - protein, 12 aa, Artificial
  Cell penetration peptide derived from Tat protein
RKKRRQRRRAHQ

SEQ ID NO: 2 - protein, 12 aa, Artificial
  Cell penetration peptide derived from Tat protein
RKKRRQRRRAHQ

SEQ ID NO: 3 - protein, 17 aa, Artificial
  Cell penetration peptide derived from Antennapedia protein of Drosophila
RQRIKIWFQNRRMKWKK

SEQ ID NO: 4 - protein, 12 aa, Artificial
  Cell penetration oligopeptide derived from human clock protein
RVSRNKSEKKRR

SEQ ID NO: 5 - protein, 15 aa, Artificial
  Cell penetration peptide derived from hPer1 protein
RRHHCRSKAKRSRHH

SEQ ID NO: 6 - protein, 18 aa, Artificial
  Cell penetration peptide derived from hPer2 protein
KKTGKNRKLKSKRVKPRD

SEQ ID NO: 7 - protein, 9 aa, Artificial
  Cell penetration peptide derived from Tat protein
RKKRRQRRR

SEQ ID NO: 8 - protein, 21 aa, Artificial
  Cell penetration peptide
KETWWETWWTEWSQPKKKRKV

SEQ ID NO: 9 - DNA, 1536 bp, Artificial
  Truncated Uvde1 protein with hexahistidine tag at C-terminus
atgggacctcacaaaaaaagtacttctacgtctacacgaaagagggcacgtagcagtaaaaagaaagcgacagattctgtttccgataaaattgatgagtctgttgcgtcctatgattcttcaactcatcttaggcgatcgtcgagatcaaaaaaaccggtcaactacaattcctcgtcagaatccgaatcggaggagcaaattagtaaagctactaaaaaagttaaacaaaaagaggaagaggagtatgttgaagaagtcgacgaaaagtctcttaaaaatgaaagtagctctgacgagttcgaaccggttgtgccggaacagttggaaactccaatttctaaacgaagacggtctcgttcttctgcaaaaaatttagaaaaagaatctacaatgaatcttgatgatcatgctccacgagagatgtttgattgtttggacaaacccataccctggcgaggacgattggggtatgcttgtttgaatactattttaaggtcaatgaaggagagggttttttgttcacgcacctgccgaattacaaccattcaacgtgatgggctcgaaagtgtcaagcagctaggtacgcaaaatgttttagatttaatcaaattggttgagtggaatcacaactttggcattcacttcatgagagtgagttctgatttatttcctttcgcaagccatgcaaagtatggatatacccttgaatttgcacaatctcatctcgaggaggtgggcaagctggcaaataaatataatcatcgattgactatgcatcctggtcagtacacccagatagcctctccacgagaagtcgtagttgattcggcaatacgtgatttggcttatcatgatgaaattctcagtcgtatgaagttgaatgaacaattaaataaagacgctgttttaattattcaccttggtggtacctttgaaggaaaaaaagaaacattggataggtttcgtaaaaattatcaacgcttgtctgattcggttaaagctcgtttagttttagaaaacgatgatgtttcttggtcagttcaagatttattacctttatgccaagaacttaatattcctctagttttggattggcatcatcacaacatagtgccaggaacgcttcgtgaaggaagtttagatttaatgccattaatcccaactattcgagaaacctggacaagaaagggaattacacagaagcaacattactcagaatcggctgatccaacggcgatttctgggatgaaacgacgtgctcactctgatagggtgtttgactttccaccgtgtgatcctacaatggatctaatgatagaagctaaggaaaaggaacaggctgtatttgaattgtgtagacgttatgagttacaaaatccaccatgtcctcttgaaattatggggcctgaatacgatcaaactcgagatggatattatccgcccggagctgaaaagcgtttaactgcaagaaaaaggcgtagtagaaaagaagaagtagaagaggatgaaaaacatcaccatcaccatcactaa

SEQ ID NO: 10 - protein, 511 aa, Artificial
  Synthetic Construct
MGPHKKSTSTSTRKRARSSKKKATDSVSDKIDESVASYDSSTHLRRSSRSKKPVNYNSSSESESEEQISKATKKVKQKEEEEYVEEVDEKSLKNESSSDEFEPVVPEQLETPISKRRRSRSSAKNLEKESTMNLDDHAPREMFDCLDKPIPWRGRLGYACLNTILRSMKERVFCSRTCRITTIQRDGLESVKQLGTQNVLDLIKLVEWNHNFGIHFMRVSSDLFPFASHAKYGYTLEFAQSHLEEVGKLANKYNHRLTMHPGQYTQIASPREVVVDSAIRDLAYHDEILSRMKLNEQLNKDAVLIIHLGGTFEGKKETLDRFRKNYQRLSDSVKARLVLENDDVSWSVQDLLPLCQELNIPLVLDWHHHNIVPGTLREGSLDLMPLIPTIRETWTRKGITQKQHYSESADPTAISGMKRRAHSDRVFDFPPCDPTMDLMIEAKEKEQAVFELCRRYELQNPPCPLEIMGPEYDQTRDGYYPPGAEKRLTARKRRSRKEEVEEDEKHHHHHH

SEQ ID NO: 11 - DNA, 1575 bp, Artificial
  Truncated Uvde1 protein with Tat-derived cell penetration peptide at the N-terminus and a hexahistidine tag at the C-terminus
atgggatacggccgcaagaaacgccgccagcgccgacgcggtggacctcacaaaaaaagtacttctacgtctacacgaaagagggcacgtagcagtaaaaagaaagcgacagattctgtttccgataaaattgatgagtctgttgcgtcctatgattcttcaactcatcttaggcgatcgtcgagatcaaaaaaaccggtcaactacaattcctcgtcagaatccgaatcggaggagcaaattagtaaagctactaaaaaagttaaacaaaaagaggaagaggagtatgttgaagaagtcgacgaaaagtctcttaaaaatgaaagtagctctgacgagttcgaaccggttgtgccggaacagttggaaactccaatttctaaacgaagacggtctcgttcttctgcaaaaaatttagaaaaagaatctacaatgaatcttgatgatcatgctccacgagagatgtttgattgtttggacaaacccataccctggcgaggacgattggggtatgcttgtttgaatactattttaaggtcaatgaaggagagggttttttgttcacgcacctgccgaattacaaccattcaacgtgatgggctcgaaagtgtcaagcagctaggtacgcaaaatgttttagatttaatcaaattggttgagtggaatcacaactttggcattcacttcatgagagtgagttctgatttatttcctttcgcaagccatgcaaagtatggatatacccttgaatttgcacaatctcatctcgaggaggtgggcaagctggcaaataaatataatcatcgattgactatgcatcctggtcagtacacccagatagcctctccacgagaagtcgtagttgattcggcaatacgtgatttggcttatcatgatgaaattctcagtcgtatgaagttgaatgaacaattaaataaagacgctgttttaattattcaccttggtggtacctttgaaggaaaaaaagaaacattggataggtttcgtaaaaattatcaacgcttgtctgattcggttaaagctcgtttagttttagaaaacgatgatgtttcttggtcagttcaagatttattacctttatgccaagaacttaatattcctctagttttggattggcatcatcacaacatagtgccaggaacgcttcgtgaaggaagtttagatttaatgccattaatcccaactattcgagaaacctggacaagaaagggaattacacagaagcaacattactcagaatcggctgatccaacggcgatttctgggatgaaacgacgtgctcactctgatagggtgtttgactttccaccgtgtgatcctacaatggatctaatgatagaagctaaggaaaaggaacaggctgtatttgaattgtgtagacgttatgagttacaaaatccaccatgtcctcttgaaattatggggcctgaatacgatcaaactcgagatggatattatccgcccggagctgaaaagcgtttaactgcaagaaaaaggcgtagtagaaaagaagaagtagaagaggatgaaaaacatcaccatcaccatcactaa

SEQ ID NO: 12 - protein, 524 aa, Artificial
  Synthetic Construct
MGYGRKKRRQRRRGGPHKKSTSTSTRKRARSSKKKATDSVSDKIDESVASYDSSTHLRRSSRSKKPVNYNSSSESESEEQISKATKKVKQKEEEEYVEEVDEKSLKNESSSDEFEPVVPEQLETPISKRRRSRSSAKNLEKESTMNLDDHAPREMFDCLDKPIPWRGRLGYACLNTILRSMKERVFCSRTCRITTIQRDGLESVKQLGTQNVLDLIKLVEWNHNFGIHFMRVSSDLFPFASHAKYGYTLEFAQSHLEEVGKLANKYNHRLTMHPGQYTQIASPREVVVDSAIRDLAYHDEILSRMKLNEQLNKDAVLIIHLGGTFEGKKETLDRFRKNYQRLSDSVKARLVLENDDVSWSVQDLLPLCQELNIPLVLDWHHHNIVPGTLREGSLDLMPLIPTIRETWTRKGITQKQHYSESADPTAISGMKRRAHSDRVFDFPPCDPTMDLMIEAKEKEQAVFELCRRYELQNPPCPLEIMGPEYDQTRDGYYPPGAEKRLTARKRRSRKEEVEEDEKHHHHHH